(19) United States
(12) Patent Application Publication
(10) Pub. No.: US 2010/0004655 A1
(43) Pub. Date: Jan. 7, 2010

(54) APPARATUS FOR PERCUTANEOUS DELIVERY OF BONE-FILLING MATERIAL

(75) Inventor: Gil-Woon Choi, Anyang (KR)

Correspondence Address: EDWARDS ANGELL PALMER & DODGE LLP, P.O. BOX 55874, BOSTON, MA, 02205, US

(73) Assignee: Taeyeon Medical Co., Ltd., Incheon (KR)

(21) Appl. No.: 12/263,655

(22) Filed: Nov. 3, 2008

(30) Foreign Application Priority Data
Jul. 3, 2008 (KR) 10-2008-0064348
Jul. 29, 2008 (KR) 10-2008-0074198

(51) Int. Cl. A61B 17/58

(52) U.S. Cl. 606/93

(57) ABSTRACT

The present invention relates to an apparatus for percutaneous delivery of a bone-filling material, which comprises a cannula including a hollow main pipe, a handle attached to an end of the main pipe and having a hole connected to with the inside of the main pipe, and a branch pipe providing a passage for a material to be inserted into the interior of the main pipe. The apparatus has an improved functional structure to satisfy an operator and a surgical patient by improving conveniences during and after operation.

### CROSS-REFERENCE TO RELATED APPLICATION

This application claims under U.S.C. §119(a) on Korean Patent Application Nos. 10-2008-0064348 filed on Jul. 3, 2008 and 10-2008-0074198 filed on Jul. 29, 2008, the entire contents of which are incorporated herein by reference.

### BACKGROUND

(a) Technical Field

The present disclosure relates to an apparatus for percutaneous delivery of bone-filling material, in particular an apparatus comprising a cannula equipped with a branch pipe for inserting a material into the interior of the cannula.

(b) Background Art

Vertebroplasty is a medical procedure where the original height and angle of kyphosis of a fractured vertebra are restored, followed by its stabilization using injected bone filler material. The procedure is commonly done percutaneously. Height and angle restorations are carried out by using either hydraulic or mechanical intravertebral expansion.

Restorators and accessories are known as apparatuses used for vertebroplasty. A cavity-forming instrument, such as a balloon catheter, is used together with a restorator in case of balloon kyphoplasty. Instruments that are used for providing a path to a bone each comprises a needle generally composed of a pipe and a steel wire, a wire pin, a cannula, and an expander. Further, they may additionally comprise a spacer, a filler, a pusher, etc.

The balloon kyphoplasty is performed by, for example, inserting a long thin special tube into a compression fractured region, inserting a balloon through the tube and expanding it to a normal height, and then removing the balloon and filling the thus-formed space with a bone-filling material (e.g. bone cement and bone substitute).

The procedures of the conventional kyphoplasty can be known from the descriptions of U.S. Pat. No. 6,241,734, which is incorporated herein by reference. In detail, the balloon kyphoplasty is performed by: inserting a needle with a pointed tip (in an operation) into the vertebral body through surgical cut from the outside of the body and removing the steel wire out of the pipe of the needle; thereafter, inserting a wire pin into the pipe and pulling the pipe out of the body; thereafter, inserting a cannula and an expander into the body with the guidance by the wire pin and expanding the inside of the vertebral body by operating the expander from the outside, and then separating the expander from the body; thereafter, inserting a spacer into the cannula and ensuring an inner space using a drill-shaped tip such that cavity-forming instruments, such as a balloon catheter, can be smoothly inserted; thereafter, equipping a restorator with a balloon catheter and inserting the tip of the catheter with a balloon into the cannula, and then expanding the balloon inserted into the inside of the vertebral body using pressure from the restorator; thereafter, repeatedly inserting each of a plurality of fillers (disposal) into the cannula and injecting the bone-filling material into the expanded space inside the vertebral body by pushing the inside of the pipe-shaped filler using a pusher, whereby the operation is completed.

However, the catheter accessories that are used in the surgical operation in the related art have some problems and inconvenience.

For example, the needle that is inserted into the surgical patient's body through the skin by an operator is commonly formed of a probe with a pointed tip and inserted through the vertebral body by a surgical hammer or an electric tool. However, after being inserted to the vertebral body, the needle is difficult to be separated from the bone because the compounds of inorganic substances and organic substances act like a concrete. In order to overcome this problem, atypical methods such as using a specific separating device or hitting the needle in the opposite direction to the hitting direction to be inserted are used, which is inconvenient for the operator and reduces operational accuracy.

Further, according to the procedure, three instruments of a filler, a cannula, and a pusher are used in the injection of a bone-filling material. That is, the injection of a bone-filling material is performed in three steps: filling cement into the filler, inserting the filler into the cannula, and pushing the filler with the pusher. Since the filler, an instrument that is inserted into the cannula, has a smaller diameter than the cannula, a plurality of fillers was needed to fill a sufficient amount of bone-filling material (e.g. cement or bone-substitute) into the ensured space. Further, while waiting an operation after filling the cement into the filler, the cement may harden such that it is difficult to insert the cement. Accordingly, management of time and careful use were required and a limited amount of fillers were provided during the limited time, such that cement would not be sufficiently injected.

The above information disclosed in this Background section is only for enhancement of understanding of the background of the invention and therefore it may contain information that does not form the prior art that is already known in this country to a person of ordinary skill in the art.

### SUMMARY

An object of the invention is to provide an apparatus for percutaneous delivery of a bone-filling material that reduces various disadvantageous effects due to complex components, by simplifying the components for the procedure of inserting a bone-filling material and thereby reducing time for insertion. Another object of the invention is to provide an apparatus that makes it easier to insert the bone-filling material into the cannula and accurately adjust the speed and amount of insertion. Yet another object of the invention is to easily separate the bone-filling material inserted in a cavity of the bone from the cannula.

In order to achieve the objects of the invention, in one aspect, the present invention provides an apparatus for percutaneous deliver of a bone-filling material, which comprises a cannula including: a hollow main pipe; a handle attached to an end of the main pipe and having a hole connected to the inside of the main pipe; and a branch pipe providing a passage for a material to be inserted into the interior of the main pipe.

In another aspect, the present invention provides an apparatus for percutaneous deliver of a bone-filling material, comprising: a needle assembly for forming a path from a skin to a bone; a guiding instrument formed of a wire pin that is to be inserted into the needle assembly; a cannula comprising a hollow main pipe, a handle attached to an end of the main pipe and having a hole connected with the inside of the main pipe, and a branch pipe providing a passage for inserting a material into the interior of the hollow main pipe, the cannula being able to be inserted into the path formed by the needle assembly (100); an expander assembly comprising an expanding pipe and a head which can be fitted into the handle of the cannula; a cavity-forming instrument that is to be inserted into the bone through the cannula after the expander assembly inserted in the cannula is separated, thereby forming a space inside the bone; a cap that is to be fitted into the handle of the cannula after the cavity-forming instrument inserted into the cannula is separated; an instrument for injecting a bone-filling material into the main pipe of the cannula via the branch pipe of the cannula after the cannula is closed by the cap; and a pusher for pushing to the bone the bone-filling material inserted in the main pipe of the cannula, wherein the cannula is inserted with the guidance of the guiding instrument after the needle assembly is separated from the guiding instrument.

According to the present invention, the needle and the expander are easily separated from the bone, and the spacer does not incur unnecessary cutting. Since the bone-filing material is injected into the interior of the main pipe through the branch pipe which is used as a path for inserting the bone-filling material into the main pipe, the operation is easier and the procedure is simplified and quickly performed as compared with the prior art. Further, since the pusher of the invention has a larger length than the cannula and has the recessed end, the pusher cuts cement in the final push when pushing the cement inserted in the cannula.

Also, since the bone-filling material is inserted into the cannula itself, it has the following advantages as compared with when a filler filled with a bone-filling material is inserted into a cannula: (i) it is possible to simplify the components because it does not use a filler; (ii) it is possible to inject a larger amount of bone-filling material for the same length of cannula because the inner diameter of the cannula is larger than the inner diameter of the filler; and (iii) it is possible to reduce the time required for inserting the bone-filling material into the filler and waiting an operation. Further, when bone cement is used as a bone-filling material, it is possible to accurately inject the cement before the bone cement becomes hardened.

Further, since the threaded-plunger is provided and the knob is provided at an end of the threaded-plunger, and the plunger can be moved forward in the tube of the injector by rotating the knob, it is possible to accurately adjust the insertion speed and the insertion amount of the material. Further, when the nozzle is disposed between the injecting instrument and the branch pipe, it is possible to easily ensure a working space and sight for inserting the material.

### BRIEF DESCRIPTION OF THE DRAWINGS

The above and other features and advantages of the present invention will become more apparent by describing in detail preferred embodiments thereof with reference to the attached drawings in which:

FIG. 1 is a perspective view of a needle assembly, a guiding instrument, a cannula, an expander assembly, a spacer assembly, a cap, an injecting instrument, and a pusher assembly according to an embodiment of the invention;

FIG. 2 is a perspective view of a needle assembly in a combined state of an apparatus according to an embodiment of the invention;

FIG. 3 is a perspective view of a needle assembly of an apparatus according to an embodiment of the invention;

FIG. 4 is a perspective view of a needle assembly in a separated state of an apparatus according to an embodiment of the invention;

FIG. 5 is a perspective view of a cannula and an expander in a combined state of an apparatus in a combined state according to an embodiment of the invention;

FIG. 6 is an exploded view of a cannula, an expander, and a guiding instrument of an apparatus according to an embodiment of the invention;

FIG. 7 is a perspective bottom view of the configuration of a coupling portion of a steel wire assembly and an expander assembly;

FIG. 8 is a schematic view illustrating a coupling portion in a combined state;

FIG. 9 is a schematic view illustrating a coupling portion in a separated state;

FIG. 10 is an enlarged view of the front end of a spacer of an apparatus according to an embodiment of the invention;

FIG. 11 is a view showing an injector according to an embodiment of the invention;

FIG. 12 is a view showing attachment of an injector for injecting a bone-filling material into a branch pipe of a cannula, in which the upper portion of the cannula is closed by a cap and the attachment is made by thread-fastening;

FIG. 13 is a schematic view of a cannula according to an embodiment of the invention, in which a branch pipe penetrates a handle; and

FIG. 14 is a view illustrating the operation of a pusher.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

As discussed above, in one aspect, the present invention provides an apparatus for treating a bone that comprises a cannula providing a path from a skin to the bone, in which the cannula may comprise a hollow main pipe, a handle provided at an end of the main pipe and having a hole connected to the interior of the main pipe, and a branch pipe having a passage for inserting a material into the interior of the hollow main pipe. The branch pipe may be formed to branch off from the main pipe or to be connected with a hole of the handle.

The apparatus of the invention may further comprise, in addition to the cannula, an instrument for inserting a bone-filling material through the branch pipe. It may further comprise a pusher for pushing the bone-filling material inserted into the cannula through the branch pipe to the bone. It also may further comprise a cap for closing an inlet of the handle to thereby prevent the bone-filling material inserted through the branch pipe from leaking out through the inlet of the handle.

According to the invention, since the bone-filling material is inserted into the cannula itself through the branch pipe, the configuration is simpler as compared with prior art apparatuses in which the bone-filling material is filled into a separate filler and then said filler is inserted into a cannula. Further, a cannula having a smaller outer diameter than the prior art apparatuses can be used for the same amount of a bone-filling material to be injected into the interior of the cannula; and when the same sized cannula is used, more amount of a bone-filling material can be injected at one time for the same length of the cannula, because the inner diameter of the cannula is larger than the inner diameter of the filler in the prior art apparatuses. Furthermore, since a bone-filling material is directly inserted into the cannula, the time for inserting the bone-filling material into a separate filler and waiting time for an operation can be reduced. In addition, when bone cement is used as a bone-filling material, it is possible to accurately inject the cement in accordance with the hardening speed of the bone cement. Further, since a bone-filling material is inserted through the branch pipe, a space restriction problem that may occur when using a material injecting instrument such as an injector can be prevented.

Although the apparatuses of the invention are described with reference to vertebroplasty, they also can be applied to other various types of procedures involving injection of a bone-filing material.

An apparatus for percutaneous delivery of a bone-filling material according to an embodiment of the invention may comprise a needle assembly, a guiding instrument 200, a cannula 300, an expander assembly 400, a cavity-forming instrument, a cap 600, an injecting instrument for inserting a bone-filling material into the cannula 300 and a pusher 800.

The needle assembly 100 is used to form a passage from a skin to a bone. The guiding instrument 200 is formed of a wire pin that is to be inserted into the needle assembly 100. The cannula 300 is to be inserted into the passage formed by the needle assembly 100. The cannula 300 may comprise a hollow main pipe 330, a handle 311 attached to an end of the main pipe 330 and having a hole connected to the inside of the main pipe, and a branch pipe 340 providing a path for inserting a material into the hollow main pipe. The expander assembly 400 includes a head 421 which can be combined with the handle 311 of the cannula 300 and an expanding pipe 430. The cavity-forming instrument is to be inserted into the bone through the cannula 300 to form a space inside the bone after the expander assembly 400 inserted in the cannula 300 is separated. Preferably, the cavity-forming instrument may be a spacer 500 and/or a balloon catheter. The cap 600 can be combined with the handle 311 of the cannula 300 after the spacer 500 is separated from the cannula 300. The instrument for inserting a bone-filling material is to insert the bone material to the cannula 300, which is closed by the cap 600, via the branch pipe 340. The pusher 800 includes a handle 811 and is to push the bone-filling material inserted in the cannula 300 to the bone. During an operation, the cannula may be guided by the guiding instrument 200 and then inserted into the space inside the bone (e.g., vertebral body) after the needle assembly 100 is separated from the guiding instrument 200.

Preferably, the injecting instrument for inserting the bone-filling material into the cannula 300 through the branch pipe 340 may be injectors 700, 700′ and the branch pipe 340 may be connected directly or through a nozzle 770 to the injectors 700, 700′. The branch pipe 340 may be provided with a engaging portion 342 that can engage with tubes 720, 720′ of the injectors 700, 700′ or the nozzle 770, and the tubes 720, 720′ of the injectors 700, 700′ or the nozzle 770 may be provided with engaging portion 722, 722′, 772 corresponding to the engaging portion 342 of the branch pipe. It is preferable that the injectors 700, 700′ or the nozzle 770 is thread-fastened to the branch pipe 340.

The injectors may be any types of injectors. For example, a syringe that operates by manually pushing a plunger may be used. It is preferable to use an injector with a threaded-plunger. The threaded-plunger may be moved forward in the tube of the injector by thread-fastening between the plunger and the tube, or may be moved forward by thread-fastening between the plunger and a separate component connected with the tube of the injector. It is preferable for the tube of the injector to have markings, which indicate the amount of the bone-filling material inserted in the cannula. According to an embodiment, the injector 700′ may include the tube 720′ that will be filled with a material to be injected, a handle 740′ connected with the tube 720′, and a plunger 750′ thread-fastened through the handle 740′, in which the plunger 750′ has a knob 751′ at the rear end thereof and is moved forward inside the tube 720′ by thread-fastening as the knob 751′ is turned. The handle 740′ allows an operator to easily grip the injecting instrument. The knob 751′, which pushes the plunger 750′ as it is turned, enables precise control of an insertion speed and an amount of the injected material as compared with an injector using a pushing force.

It is preferable for the pusher 800 to have a length such that it protrudes out of the cannula 300 when being completely advanced inside the cannula 300. A proper length of the protrusion is 2 to 4 mm. According to this configuration, the pusher 800 can completely push the inserted bone-filling material out of the cannula 300. Suitably, the tip may be recessed, which allows the pusher to further accurately push the bone-filling material inserted in the cannula 300 and also allows the bone-filling material to cut off from the cannula 300 at the final pushing.

In an embodiment, the apparatus may further comprise a needle assembly 100 that is to establish the initial path from the skin to the bone.

Preferably, the needle assembly 100 may comprise a pipe assembly 110 and a steel wire assembly 120. The pipe assembly 110 comprises a pipe 130 and a handle 111 connected to an end of the pipe 130 and having a receiving groove 112. The steel wire assembly 120 includes a steel wire 122 that is able to be inserted into the pipe 130 of the pipe assembly 110 and a head 121 that is connected to an end of the steel wire 122 and is configured to be received in the receiving groove 112 through a coupling means.

Suitably, the coupling portion may further comprise a guiding protrusion 113 protruding from a portion of the inner side of the receiving groove 112 and a seat 129 formed on a portion of the bottom of the head 121 to lock the guiding protrusion 113.

Preferably, the coupling portion may further comprise a locking projection 128 protruding from a portion of the bottom of the head 121 to prevent the guiding protrusion 113 from being unlocked from the seat 129.

Suitably, the coupling portion may further comprise a guiding means that facilitates the guiding protrusion 113 to be locked into the seat 129.

The guiding means may be any means known in the art. According to an embodiment, the guiding means comprises a fixing groove 115 formed at the sides of the receiving groove 112 and a fixing protrusion 123 protruding from the sides of the head 121 and fitted into the fixing groove 115.

Further, the guiding means may further comprises a guide slope 127 that is formed at a predetermined angle on a portion of the bottom of the head 121 to facilitate the guiding protrusion 113 to be slid to the seat 129.

As described above, the needle assembly may suitably comprise the pipe combined with the steel wire by the coupling portion and the receiving groove, such that the needle or the steel wire in the needle can be easily separated from the bone and the operating time can be reduced.

According to an embodiment, the handle 111 of the pipe assembly 110 can receive the head 121 of the steel wire assembly 120 at the receiving groove 112 formed at the upper portion. Further, the handle 111 having the receiving groove 112 has an insertion hole 116 at the center portion thereof, so that the steel wire 122 of the steel wire assembly can be inserted into the pipe 130. A curved portion 114 may be formed on the bottom of the handle 111 to provide stability in the operation and a fixing member 118 may be provided to fix the pipe 130 to the handle 111. The fixing member 118 may be any one as long as it can perform the fixing function. Further, the handle 111 can be made of desired materials known in the art, for example, acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, and etc, but not limited thereto.

The pipe 130 of the pipe assembly 110 is hollow to insert the steel wire 122 of the steel wire assembly 130 and has a larger diameter than the steel wire 122. For example, the diameter of the pipe 130 is in the range of 1 to 6 mm. The pipe 130 can be made of a material known in the art, for example, stainless steel, aluminum, titanium etc, but not limited thereto. It is preferable that the frontal end (in an operation) of the pipe 130 is tapered (tapered portion 117) to be easily inserted into the bone.

The head 121 of the steel wire assembly 120 is provided with a grip 124 having a plurality of grooves at the front and the rear sides to allow the head 121 to be easily turned while preventing a hand gripping the instrument from slipping. An outer groove 125 may be formed below the grip 124 to provide a stable grip and reduce the entire weight. For example, the head may be made of acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, and etc, but not limited thereto.

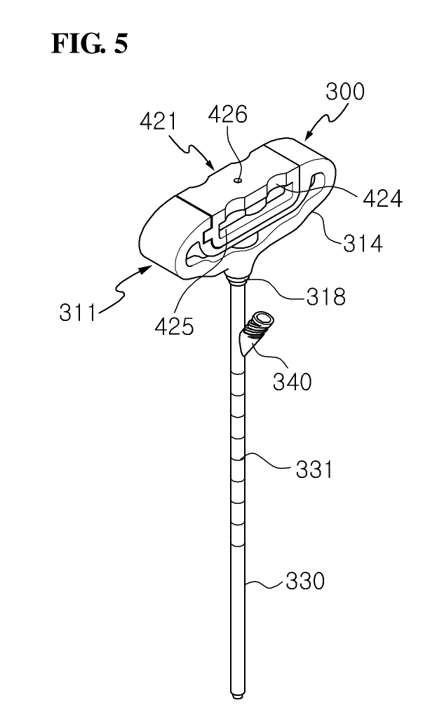

It is preferable that the frontal end (in an operation) of the steel wire 122 of the steel wire assembly 120 is sharp and pointed to be easily inserted into the bone. For example, the other end of the steel wire 122 can be in the shape of a pyramid such as triangular, quadrangular, pentagonal, or hexagonal pyramid and cheek-cut cylinder, as illustrated in FIG. 5. The steel wire 122 can be made of a material, for example, a stainless steel wire, and a titanium stick, and etc, but not limited thereto.

Preferably, the apparatus may further comprise an instrument used for a guide into the bone. An example of such instrument is the guiding instrument 200 formed of a wire pin.

According to one embodiment of the present invention, a curved portion 314 may be formed on a portion of the bottom of the handle 311 of the cannula 300. The cannula 300 may further comprise a receiving groove 312, a fixing groove 315, a guiding protrusion 313, an insertion hole 316, a fixing member 318, and a marking portion 331.

Preferably, as shown in the drawings, the receiving groove 312 may be recessed on the upper portion of the handle 311 to be combined with an expander assembly 400. The fixing grooves 315 may be formed on the sides of the handle 311 having the receiving groove 312. The guiding protrusions 313 may be formed around the side of a flat surface of the handle 311 along the receiving groove 312 to facilitate attachment and separation of the cannula 300 and the expander assembly 400. The insertion hole 316 may be provided at the center portion of the handle 311. The fixing member 318 is to fix the main pipe 330, which is joined to the lower portion of the handle 311, to the handle 311. The marking portion 331 may be provided along the length of the main pipe 330 to indicate the insertion position.

According to one embodiment, the expander assembly 400 comprise a head 421 and an extending pipe 430. The head 421 may include: a grip 424 having a plurality of grooves on the front and rear sides of the head to prevent slipping during an operation; an outer groove 425 formed at the lower end of the grip 424 to provide an improved grip and reduce the weight; a hole 426 formed through the head 421 in order that the guiding instrument 200 is capable of advancement through it; and fixing protrusions 423 formed on the left and right sides of the head 421 to fix the head to the cannula 300. The expanding pipe 430 may be tapered at the front thereof end and have a space therein, in which the guiding instrument 200 is to be inserted.

According to one embodiment, the cannula 300 suitably comprises a main pipe 330, a branch pipe 340 and a handle 311 connected to an end of the main pipe 330 and having a receiving groove 312. The expander assembly 400 suitably comprises an expanding pipe 430 which can be inserted into the main pipe 330 of the cannula 300 and a head 421 that is connected to an end of the expanding pipe 430 and is configured to be received in the receiving groove 312 through the structure of a coupling portion.

The cannula 300 can be suitably docked with the expander assembly 400, such that the operation is simplified by a one-step method and thereby the operation time is reduced.

Suitably, the coupling portion may comprise a guiding protrusion 313 that protrudes from a portion of the inner side of the receiving groove 312 and a seat 429 that is formed at a portion of the bottom of the head 421 to lock the guiding protrusion 313.

Preferably, the coupling portion may further comprise a locking projection 428 protruding from a portion of the bottom of the head 421 to prevent the guiding protrusion 313 from being unlocked from the seat 429.

Suitably, the coupling portion may further comprise a guiding means for facilitating the guiding protrusion 313 to be locked into the seat 429. The guiding means may be any means known in the art. An example of the guiding means comprises a fixing groove 315 formed at a side portion of the receiving groove 312 and a fixing protrusion 423 protruding from a side portion of the head 421 so as to fit into the fixing groove 315.

The guiding means may further comprise a guide slope 427 that is formed at a predetermined angle on a portion of the bottom of the head 421 to facilitate the guiding protrusion 313 to be slid into the seat 429.

According to the apparatuses, the cannula is docked with the expander by the coupling means, such that the expander can be easily and stably fixed to the cannula during the operation and also easily separated from the cannula or a bone.

In an embodiment, the handle 311 of the cannula 300 can receive the head 421 of the expander at the receiving groove 312 which is formed at a upper portion. Further, the handle 311 having the receiving groove 312 has an insertion hole 316 at the center portion thereof, so that the expanding pipe 430 can be put into the inside of the main pipe 330. A curved portion 314 may be formed on a portion of the bottom of the handle 311 to provide stability in the operation and a fixing member 318 may be provided to fix the main pipe 330 to the handle 311. The materials by which the fixing member 318 and the handle 311 are made of or from are not limited. Suitably, the handle 311 can be made of materials known in the art, for example, acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, heat resistant resin etc.

The main pipe 330 of the cannula is hollow to insert the expanding pipe 430 and has a larger diameter than the expanding pipe 430. For example, the diameter of the pipe 330 may be in the range of 2.5 to 7.5 mm. The pipe 330 can be made of any material known in the art, for example, a stainless steel, aluminum, titanium etc, but not limited thereto. A marking 331, which indicates the insertion depth of the cannula, is provided on a side of the pipe 330.

According to an embodiment of the invention, the head 421 of the expander assembly 400 may be provided with a grip 424 having a plurality of grooves at the front and rear sides to allow the head 421 to be easily turned while preventing a hand gripping of the instrument from slipping. An outer groove 425 may be formed below the grip 424 to provide a stable grip and reduce the entire weight. The head 420 may be made of, for example, acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, and etc, but not limited thereto.

Further, the expanding pipe 430 of the expander assembly 400 is hollow to insert another instrument. The expanding pipe 430 has a diameter smaller than the main pipe 330 of the cannula and larger than another instrument (e.g. guiding instrument) that can be inserted therein. It is preferable that the frontal end (in an operation) of the expanding pipe 430 is tapered to be easily inserted into the bone. The expanding pipe 430 can be made of a material, for example a stainless steel, aluminum, titanium etc, but not limited thereto.

According to an embodiment of the invention, the head 421 has a hole 426 at the center portion thereof, so that it is connected to the expanding pipe 430 to allow another instrument used as a guide to the bone to be passed through the expanding pipe 430 and the head 421. The instrument used as a guide to the bone may be an instrument known in the art, for example, a guiding instrument 200 formed of a wire pin.

Further, the apparatus may further comprise an instrument for forming an insertion space for the cavity-forming instrument inside the vertebral body.

According to one embodiment of the invention, a spacer instrument 500 is used as the instrument for forming the insertion space for the cavity-forming instrument. The spacer assembly 500, for example, comprises a steel wire 522 having a treaded portion 520 at an end thereof and being able to be inserted into the interior of the main pipe 330 of the cannula 300 and a handle 511 attached to the other end of the steel wire 522. The spacer assembly 500 is inserted into the main pipe 330 of the cannula 300 after the expander assembly 400 is undocked from the cannula 300.

Meanwhile, the cavity-forming instrument used in the invention may be any one known in the art, and for example, may be a balloon catheter having a structure known in the art.

According to an embodiment of the invention, the threaded portion 520 is formed of a double- to fourfold-thread having a phase difference of 90° to 180° such that a distance that the thread moves forward in one turn thereof is equal to two to four times the axial distance between any point on a thread and a corresponding point on the next thread. Preferably, the threaded portion 520 may be formed of a threefold-thread having the phase difference of 120° such that a distance that the thread moves forward in one turn thereof is equal to three times the axial distance between any point on a thread and a corresponding point on the next thread.

According to an embodiment, the pusher 800 may comprise: a snowman-shaped handle 811 at the upper portion thereof; and a pin 822 connected to the lower end of the handle 811 to push medical cement filled in the cannula 300.

A general operation of vertebroplasty using the apparatus is exemplified hereafter. In the first step, the surgical patient's back is cut open and the steel wire assembly of the needle assembly is inserted into the vertebral body through the subcutaneous tissue, in which a needle including a pipe is also inserted into the vertebra.

In the second step, the inserted steel wire assembly of the needle is separated from the pipe, a wire pin is inserted into the pipe and positioned in place, and the inserted pipe is removed from the vertebra such that the entire needle is separated.

In the third step, the cannula is inserted while being guided by the wire pin partially inserted in the vertebral body, in which an expander with the tapered front end is inserted into the cannula for easy insertion of the cannula.

In the fourth step, the expander and the wire pin are separated or undocked from the cannula and the spacer is inserted into the cannula to form a space such that a balloon catheter can be smoothly inserted.

In the fifth step, the catheter with a balloon at the front end (in an operation) is inserted into the vertebra through the cannula, a separate cylinder is attached to the outer side of the catheter, and then the balloon of the catheter is expanded by operating the attached cylinder.

In the sixth step, after the body of the vertebra returns to the initial shape, the balloon catheter is separated from the cannula and a cap is applied to the receiving groove of the cannula, a bone-filling material (e.g. medical bone cement) is filled into the main pipe of the cannula through the branch pipe; thereafter, the pusher is pushed into the main pipe of the cannula such that the cement is pushed into the space formed by the balloon inside the vertebral body.

As cement is injected into the fractured vertebra by the above steps and then hardened therein, the body of the vertebra can return to the initial shape.

Hereinafter, the apparatuses will be described in more detail with regard to the accompanying drawings.

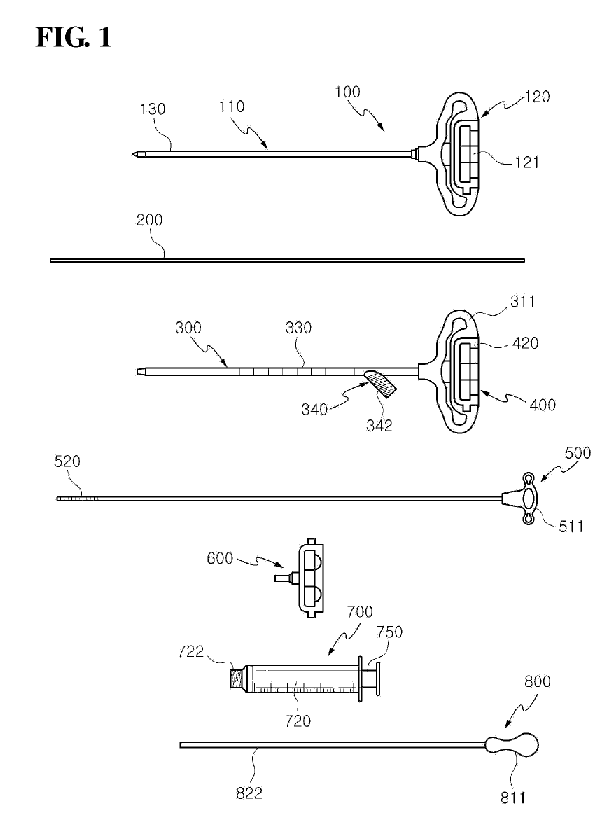

As shown in FIG. 1, an apparatus according to an embodiment may comprise a needle assembly 100, a guiding instrument 200, a cannula 300, an expander assembly 400, a spacer assembly 500, a cap 600, an injector 700, and a pusher assembly 800.

More specifically, the needle assembly 100 comprises a pipe assembly 110 and a steel wire assembly 120. The pipe assembly 10 is formed of stainless steel or aluminum and includes a pipe 130 and a handle 111. The handle 111 has a receiving groove 112 and is connected to an end of the pipe 130. The steel wire assembly 120 includes a steel wire 122 and a head 121. The steel wire 122 is to be inserted into the pipe 130 of the pipe assembly 110. The head 121 is connected with an end of the steel wire 122 and is configured to be received in the receiving groove 112 through a coupling means.

The guiding instrument 200 is, suitably, formed of a wire pin and configured to be inserted into the needle assembly 100.

The cannula 300 includes a handle 311, a main pipe 330 and a branch pipe 340. The handle is provided with a receiving groove 312. The cannula 300 is to be guided and inserted into the guiding instrument 200 after the needle assembly 100 is separated from the guiding instrument 200.

The expander assembly 400 comprises a head 421 an expanding pipe 430. The head can be received in the receiving groove 312 of the cannula 300.

The spacer 500 includes a handle 511 at the rear end thereof and a threaded-portion 520 at the front end. The handle 511 is to be inserted into the cannula 300 after the expander assembly 400 inserted in the cannula 300 is separated.

The cap 600 is provided to close the handle-sided inlet of the main pipe of the cannula after the spacer 500 inserted in the cannula 300 is separated from the cannula 300;

The injector 700 is provided to insert a bone-filling material into the main pipe of the cannula through the branch pipe.

The pusher assembly 800 comprises a pin 822 for pushing the bone-filling material filled in the main pipe and a handle 811 used for a careful operation.

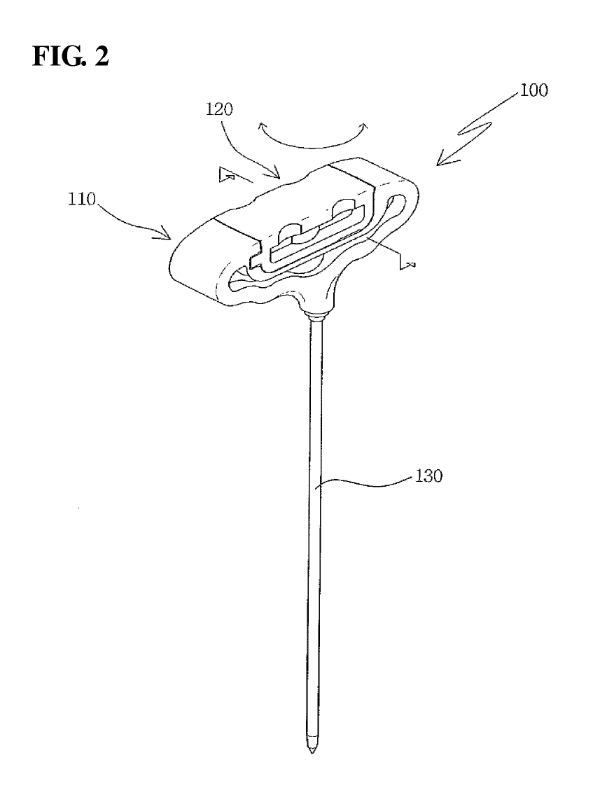

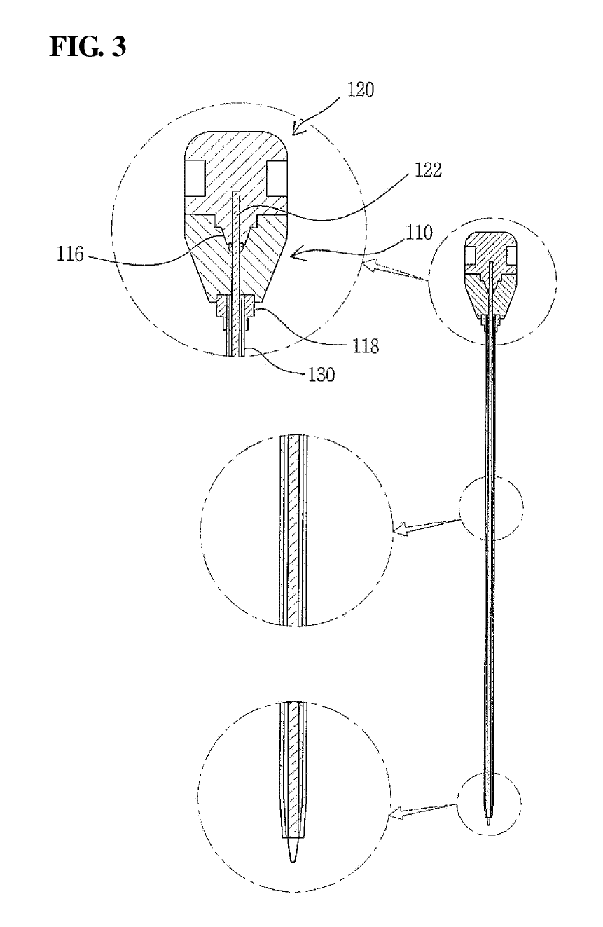

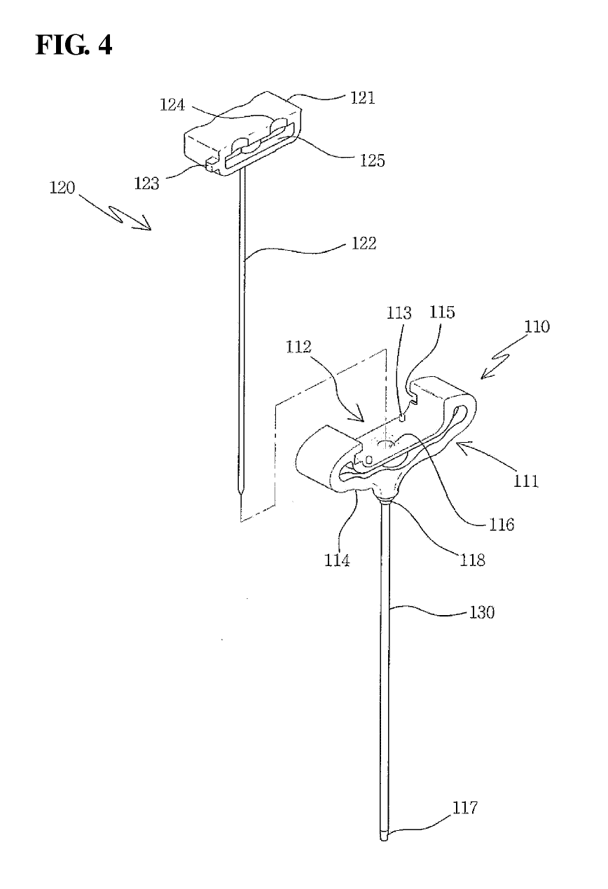

FIGS. 2 to 4 are views of a needle assembly 100 according to an embodiment of the invention.

Referring to FIGS. 2 to 4, the needle assembly 100 comprises: a pipe assembly 110 comprising a pipe 130 and a handle 111 connected to an end of the pipe 130 and having a receiving groove 112; and a steel wire assembly 120 comprising a steel wire 122 that is configured to be inserted into the pipe 130 of the pipe assembly 110 and a head 121 that is connected to an end of the steel wire 122 and is configured to be received in the receiving groove 112 through a coupling means.

The handle 111 of the pipe assembly 110 has a receiving groove 112 at a upper portion thereof to receive the head 121 of the steel wire assembly 120, and a curved portion 114 at a bottom portion. Fixing grooves 115 are formed at both sides of the receiving groove 112. Fixing protrusions 123 are formed at the head 121 of the steel wire assembly 120, which are fitted into the fixing grooves 115. Further, in a plan view, guiding protrusions 113 are formed on the inner side of the receiving groove 112 to easily dock/undock the pipe assembly 110 with/from steel wire assembly 120 and an insertion hole 116 is formed at the center portion of the handle 111 having the receiving groove 112 to insert an a wire 122 of the steel wire assembly into a pipe 130. The pipe 130 is fixed to the bottom of the handle 111 by a fixing member 118 and the pipe 130 has a tapered portion 117 at the frontal end (in an operation).

The steel wire assembly 120 includes the head 121 and the steel wire 122. Similar to the curved portion 114 formed to provide stability when operating the pipe assembly, the head 121 has a grip 124 having a plurality of grooves at the front and rear sides to allow a hand gripping the instrument to easily turn the instrument without slipping. An outer groove 125 is formed at the lower portion of the grip 124 to provide a stable grip and reduce the weight of the head. The fixing protrusions 123 are formed at the left and right sides of the head 121 and fitted into the fixing grooves 115 of the pipe assembly 110. The front end of the steel wire 122 is sharp and pointed in the shape of a cheek-cut cylinder or a pyramid, to be easily inserted into the vertebral body.

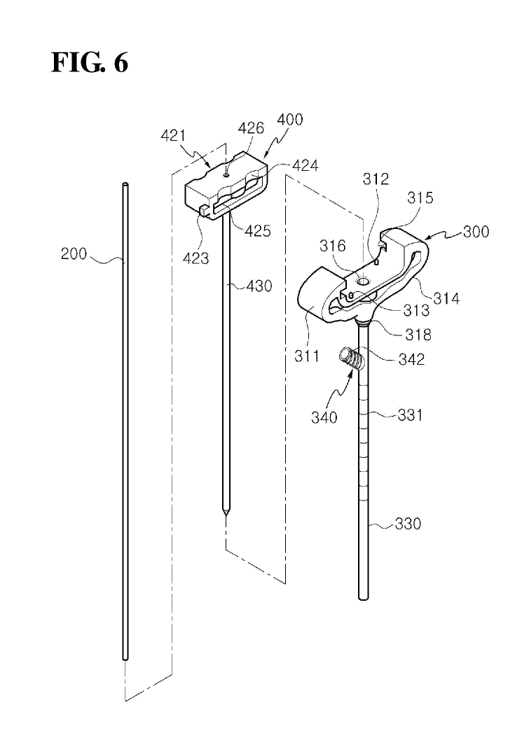

FIGS. 5 and 6 are views of a cannula and an expander assembly according to an embodiment of the invention.

The cannula 300 includes a handle 311 having a receiving groove 312 on an upper portion thereof to receive a head 421 of an expander assembly 400, and a curved portion 314 at a bottom portion thereof. Fixing grooves 315 are formed at both sides of the receiving groove 312. Fixing protrusions 423 are formed at the head 421 of the expander assembly 400, which are fitted into the fixing grooves 315. Further, in a plan view, a guiding protrusion 313 is formed on the inner side of the receiving groove 312 to easily dock/undock the cannula 300 with/from the expander assembly 400 and an insertion hole 316 is formed at the center portion of the handle 311 having the receiving groove 312 to insert an expanding pipe 430 of the expander assembly into a main pipe 330 of the cannula 300. The main pipe 330 is fixed to the bottom of the handle 311 by a fixing member 318, a marking portion 331 is provided along the length of the main pipe 330 to indicate the insertion position, and a branch pipe 340 is disposed, as a path through which a material is injected into the main pipe 330, at the upper portion of the main pipe 330.

Further, the expander assembly 400 includes the head 421 and the expanding pipe 430. Similar to the curved portion 314 formed to provide stability when operating the cannula 300, the head 421 has a grip 424 having a plurality of grooves at the front and the rear sides to allow a hand gripping the instrument to easily turn the instrument without slipping. An outer groove 425 is formed below the grip 424 to provide a stable grip and reduce the weight of the head. The fixing protrusions 423 are formed at the left and right sides of the head 421 and fitted into the fixing grooves 315 of the cannula 300. Further, the head 421 has a hole 426 at the center portion thereof to be connected with the expanding pipe 430 such that a guiding instrument 200 can pass through the expanding pipe 430 and the head 421. The expanding pipe 430 is hollow to allow the guiding instrument 200 to slip into the inside and is tapered at the frontal end.

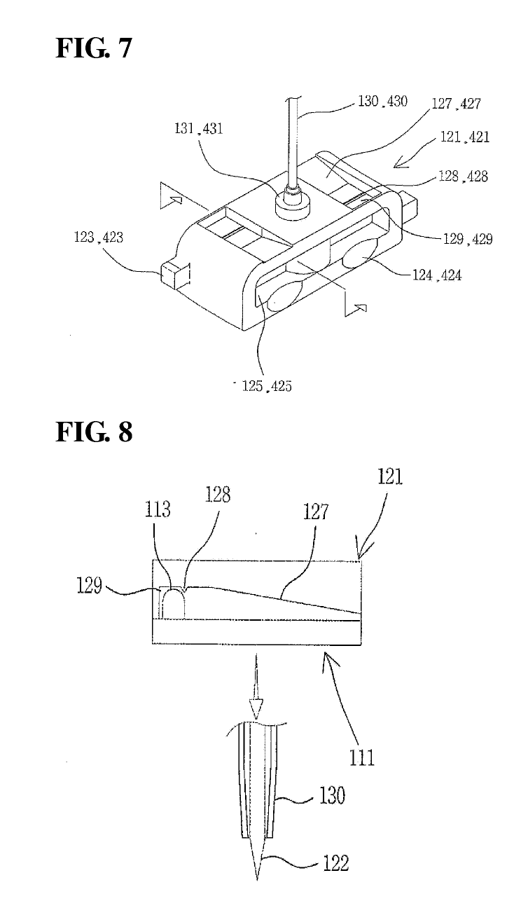

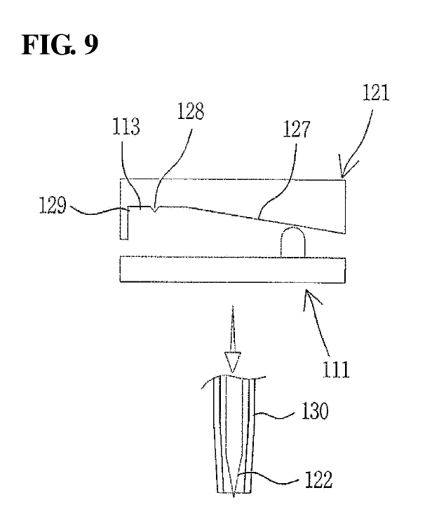

FIG. 7 is a perspective bottom view illustrating the configuration of a coupling portion that is a portion of the steel wire assembly 120 or the expander assembly 400, and FIGS. 8 and 9 are views showing that the coupling portion of a needle assembly where the pipe assembly and the steel wire assembly are combined/docked together (FIG. 8) and separated/undocked from each other (FIG. 9).

The steel wire assembly 120 and the expander assembly 400, as shown in FIG. 7, each have, at their bottoms: guide slopes 127, 427 that guide the guiding protrusions 113, 313 of the pipe assemblies 110 to slide toward seats 129, 429 to be docked/combined with the pipe assembly 110 or the cannula 300; locking projections 128, 428 that prevent the guiding protrusions 113, 313, that lead the docking through a moving along the guide slopes 127, 427, from unlocking by a moving backward; and the seats 129, 429 where the guiding protrusions 113, 313 passing the locking projections 128, 428 are seated. With this structure, it is possible to easily separate the steel wire from the needle or the bone, and stably combine or dock the expander with the cannula and easily separate them.

An exemplary operation of the guiding protrusions 113, 313, the guide slopes 127, 427, and the locking projections 128, 428 are illustrated in FIGS. 8 and 9, with reference to the needle assembly 100.

As shown in FIG. 8, when the guiding protrusion 113 of the pipe assembly 110 is guided along the guide slopes 127 of the steel wire assembly 120 and pass the locking projections 128, it is placed on the seat 129 of the steel wire assembly 120 and the docking of the pipe assembly 110 and the steel wire assembly 120 is completed and the steel wire 122 of the steel wire assembly 120 becomes to protrude out of the pipe 130 of the pipe assembly 110 as illustrated in the lower part of FIG. 8. With this structure, the steel wire assembly 120 can perform the initial establishment of a path to the vertebral body.

In order to pull the steel wire 122 run down to the bone out of the vertebral body, the operation illustrated in FIG. 9 is performed. That is, as an operator takes hold of and turns the grip 124 of the steel wire assembly 120 docked with the pipe assembly 110, the guiding protrusion 113 is moved along the guide slopes 127 and the steel wire assembly 120 is separated /undocked from the pipe assembly 110. Further, the steel wire 122 of the steel wire assembly 120 is pulled out of the vertebral body by moving it upward out of the pipe 130 of the pipe assembly 110.

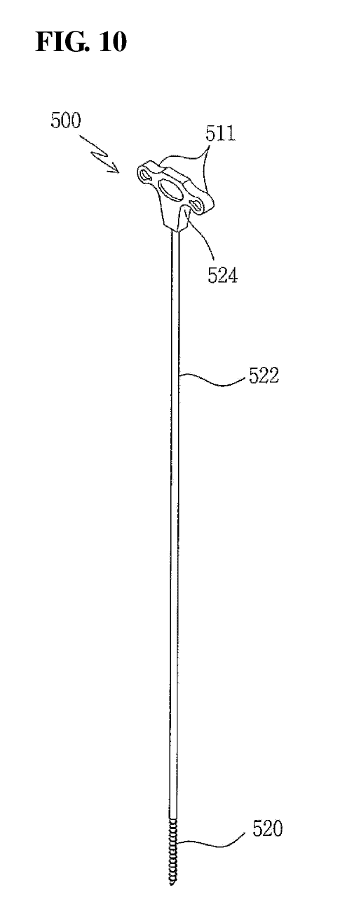

FIG. 10 is an enlarged view showing the structure of a spacer assembly 500 according to an embodiment of the invention. The space assembly 500, as described above, is provided to form a space to smoothly insert a cavity-forming instrument, such as a balloon catheter, into the vertebra.

The space instrument 500 includes a handle 511 and a steel wire 522. The handle 511 has protrusions, which are bilaterally symmetric, at the upper portion and a curved grip 524 at the bottom thereof. According to this structure, an operator can easily twist the grip of the hand taking hold of the handle 511 with fingers in the grip 524. The steel wire 522 is connected to the lower end of the handle 511 with the grip 524. A threaded portion 520 is formed at the front end (in an operation) of the steel wire 522 to form a space inside the bone. In order not to cause excessive cutting that may occur when using the drill-bit type in the related art, the threaded portion 520 is formed of a triple-thread such that it has a phase difference of 120° and the axial distance that the thread moves in one turn around the screw is equal to three times the axial distance between two points corresponding to the adjacent threads.

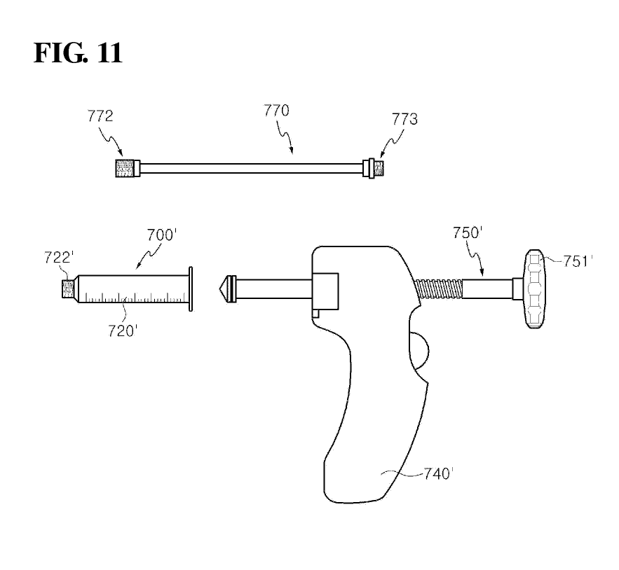

FIG. 11 shows an embodiment of an injecting instrument of an apparatus of the invention. An injector 700′ may comprise a tube 720′, a handle 740′, and a plunger 750′ thread-fastened through the handle 740′. The tube 720′ is combined with the handle 740′ when operating and the plunger 750′ can move forward in the tube 720′ as a knob 751′ at the rear end of the plunger 750′ is turned (rotated). A nozzle 770 may be disposed between an injector and a branch pipe, and in this case the one end 773 of the nozzle 770 engages with the tube 720′ at the engaging portion 722′ of the tube 720′.

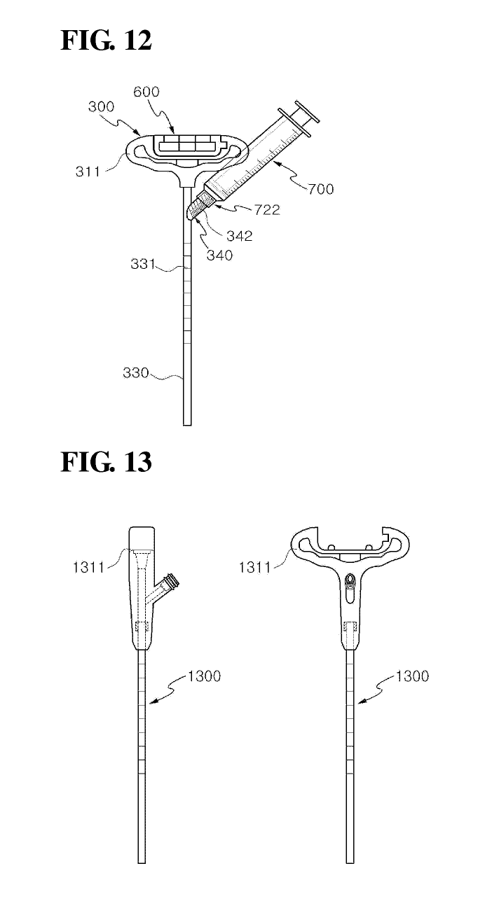

FIG. 12 shows an example of attachment of an injector for injecting a bone-filling material to a cannular through the branch pipe of the cannula. The attachment can be made by any known methods. FIG. 12 illustrates threaded-fastening attachment.

FIG. 13 shows a side view and a front view of a cannula according to an embodiment. In this embodiment, a portion of a main pipe 1300 and a branch pipe of the cannula are accommodated within a handle 1311. In a modified embodiment, though not shown, the branch pipe may be connected to a hole of the handle, not directly connected to the main pipe.

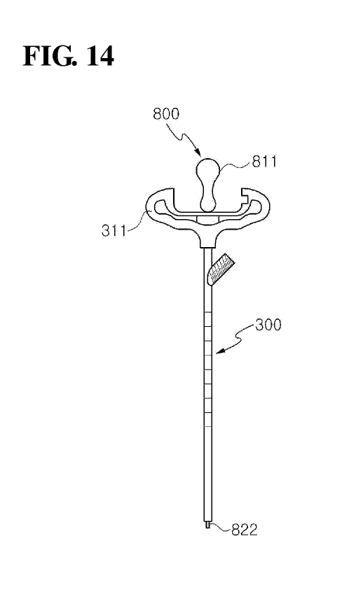

FIG. 14 illustrates the operation of a pusher. The pusher 800 comprises a snowman-shaped handle 811 at the upper portion thereof and a pin 822 connected to the lower end of the handle 811. A bone-filing material (e.g., cement) inserted in the main pipe 330 of the cannula 300 can be pushed out of the main pipe 330 into a space formed within a bone according as the pin 822 pushes the bone-filling material. When the pusher 800 is completely inserted in the cannula 300, the pusher 800 protrudes out of the end of the main pipe 330 and the bone-filing material residing in the main pipe is completely pushed out. The end of the pusher 800 may have a recessed shape to accurately push and cut off the bone-filing material.

The present apparatuses having improved functional structures satisfy an operator and a surgical patient by improving conveniences during and after operation.

What is claimed is:

1. An apparatus for treating a bone which comprises a cannula (300) providing a path from a skin to a bone, wherein the cannula (300) comprises:
a hollow main pipe (330);
a handle (311) attached to an end of the main pipe (330) and having a hole connected to the inside of the main pipe (330); and
a branch pipe (340) providing a passage for a material to be inserted into the interior of the main pipe (330).

2. The apparatus as set forth in claim 1, wherein the branch pipe (340) branches off from the main pipe (330).

3. The apparatus as set forth in claim 1, wherein the branch pipe (340) is connected with a hole of the handle.

4. The apparatus as set forth in claim 1, further comprising an instrument for inserting a bone-filling material through the branch pipe (340).

5. The apparatus as set forth in claim 1, further comprising a pusher (800) for pushing toward a bone a bone-filling material inserted in the cannula (300) through the branch pipe (340).

6. The apparatus as set forth in claim 1, further comprising a cap (600) for closing an inlet provided at the handle (311) of the cannula (300).

7. An apparatus for percutaneous deliver of a bone-filling material, comprising:
a needle assembly (100) for forming a path from a skin to a bone;
a guiding instrument (200) formed of a wire pin that is to be inserted into the needle assembly (100);
a cannula (300) comprising a hollow main pipe (330), a handle (311) attached to an end of the main pipe (330) and having a hole connected with the inside of the main pipe (330), and a branch pipe (340) providing a passage for inserting a material into the interior of the hollow main pipe (330), the cannula (300) being able to be inserted into the path formed by the needle assembly (100);
an expander assembly (400) comprising an expanding pipe (430) and a head (421) which can be fitted into the handle (311) of the cannula (300);
a cavity-forming instrument that is to be inserted into the bone through the cannula (300) after the expander assembly (400) inserted in the cannula (300) is separated, thereby forming a space inside the bone;
a cap (600) that is to be fitted into the handle (311) of the cannula (300) after the cavity-forming instrument inserted into the cannula (300) is separated;
an instrument for injecting a bone-filling material into the main pipe (330) of the cannula (300) via the branch pipe (340) of the cannula (300) after the cannula (300) is closed by the cap (600); and
a pusher (800) for pushing to the bone the bone-filling material inserted in the main pipe (330) of the cannula (300),
wherein said cannula (300) is inserted with the guidance of the guiding instrument (200) after the needle assembly (100) is separated from the guiding instrument (200).

8. The apparatus as set forth in claim 4, wherein the instrument for injecting the bone-filling material into the main pipe (330) of the cannula (300) via the branch pipe (340) is an injector (700, 700′).

9. The apparatus as set forth in claim 8, wherein the branch pipe (340) can be connected directly or through a nozzle (770) to the injector (700, 700′).

10. The apparatus as set forth in claim 8, wherein the injector (700′) comprises:
a tube (720′) for holding a material to be injected therein;
a handle (740′) configured to be connected with the tube (720′); and
a plunger (750′) thread-fastened through the handle (740′),
wherein the plunger (750′) has a knob (751′) at an end thereof and is movable toward the tube (720′) by thread-fastening as the knob (751′) is turned.

11. The apparatus as set forth in claim 9, wherein the branch pipe (340) is provided with an engaging portion, and the injector (700, 700′) or the nozzle (770) is provided with an engaging portion (722, 722′, 772) corresponding to the engaging portion of the branch pipe (340), whereby the branch pipe (340) can be connected with the injector (700, 700′) or the nozzle (770) by engagement between the engaging portions.

12. The apparatus as set forth in claim 5, wherein the pusher (800) has a length such that the pusher (800) protrudes out of the main pipe (330) of the cannula (300) when being completely inserted in the cannula (300).

13. The apparatus as set forth in claim 12, wherein the pusher (800) protrudes out of the main pipe (330) by 2 to 4 mm, when being completely inserted in the cannula (300).

14. The apparatus as set forth in claim 12, wherein the end portion of the pusher (800), which protrudes outside the main pipe (330), has a recessed shape.
